(19) United States
(12) Patent Application Publication
(10) Pub. No.: US 2007/0036843 A1
(43) Pub. Date: Feb. 15, 2007

(54) Non-ionic non-aqueous vehicles for topical and oral administration of carrier-complexed active agents

(75) Inventors: Jane Hirsh, Wellesley, MA (US); Roman V. Rariy, Allston, MA (US); Mark W. Trumbore, Westford, MA (US); Alison Fleming, North Attleboro, MA (US); Mark Hirsh, Wellesley, MA (US)

Correspondence Address: PATREA L. PABST;PABST PATENT GROUP LLP, 400 COLONY SQUARE, SUITE 1200, ATLANTA, GA, 30361, US

(73) Assignee: Collegium Pharmaceutical, Inc.

(21) Appl. No.: 11/341,016

(22) Filed: Jan. 27, 2006

Related U.S. Application Data
(63) Continuation-in-part of application No. 11/046,608, filed on Jan. 28, 2005.
(63) Continuation-in-part of application No. 11/128,947, filed on May 13, 2005.
(60) Provisional application No. 60/648,172, filed on Jan. 28, 2005.

(51) Int. Cl. A61K 9/70; A61K 9/22

(52) U.S. Cl. 424/443; 424/468

(57) ABSTRACT

An improved controlled release composition for non-parenteral administration of active agents and other therapeutics, particularly for oral or topical administration, has been developed. The composition is made by dispersing a complex formed of an active agent bound to an ion-exchange resin or to another form of resin or carrier, in a non-ionic non-aqueous (“NINA”) vehicle. The complexes are optionally coated with one or more layers of coating material to provide a controlled pattern of release of active agent from the carrier. Replacing the usual aqueous vehicle with a NINA vehicle, such as an oil or an ointment, allows the active agent-carrier complexes, with or without coatings, to be both orally and topically administered. The compositions can be formulated as powders, liquids, liquid suspensions, gels, capsules, soft gelatin capsules, tablets, chewable tablets, topical ointments, lotions, pourable or pumpable fluids, semisolid, crushable tablets, and unit-of-use sachets or capsules for reconstitution or direct application. The combination of multiple active agents is possible with this system, in which one or more active agents are bound to particles and one or more active agents are dissolved or dispersed in the NINA vehicle. This allows the combination of two or more active agents, which are otherwise incompatible, into a single dosage form.

### CROSS-REFERENCE TO RELATED APPLICATIONS

This application claims benefit under 35 U.S.C. 119, to U.S. Provisional Application 60/648,172, entitled “Non-Ionic, Non-Aqueous Vehicles for Topical and Oral Administration of Carrier-Complexed Active Agents” filed Jan. 28, 2005 by Jane Hirsh, Roman V. Rariy, Mark W. Trumbore, and Mark Hirsh and is a continuation-in-part of U.S. Ser. No. 11/046,608 entitled “Improved Dosage Forms Using Drug-Loaded Ion Exchange Resins”, filed Jan. 28, 2005 by Jane Hirsh, Alison Fleming, and Roman V. Rariy, and U.S. Ser. No. 11/128,947 entitled “Sprayable Formulations for the Treatment of Acute Inflammatory Skin Conditions”, filed May 13, 2005. by Mark Hirsh, Jane Hirsh, Ira. Skolnik, and Mark Trumbore.

### FIELD OF THE INVENTION

The present invention generally relates to a non-ionic non-aqueous (NINA) carrier for oral or topical administration of active agents complexed to ion-exchange resins or functional equivalents (“carriers”).

### BACKGROUND OF THE INVENTION

Controlled or delayed release formulations are typically in solid form. Examples include matrix systems that releases active agent over time via diffusion, enteric coated tablets, or polymer encapsulated active agents which degrade and release active agent after a period of time. It is known that common solid oral dosage forms, such as tablets or capsules, can be difficult for patients to swallow. Similarly, controlled release formulations are also likely to be difficult to swallow due to the increased bulkiness of the dosage form for a given dosage. This is particularly true for pediatric and geriatric patients, as well as for individuals who have difficulty swallowing (dysphagia) induced by disease states.

One alternative for such patients is to crush tablets or other solid dosage forms and subsequently administer them within a liquid or semi-solid vehicle. Crushing or splitting most extended or modified release solid dosage forms, however, can result in an altered release profile and is thus a potentially dangerous practice. Although some modified release formulations are known which can be sprinkled over or mixed in a semi-solid vehicle, such as coated nonpareil beads, these formulations generally have a particle diameter greater than 0.5 mm, which has an unpleasant mouth feel.

Conventional modified release tablets and capsules are also not well suited when flexible dosing is required. This is particularly an issue at the outset of treatment when the dose of an active agent is often increased slowly up to an optimal therapeutic level. Solid dosage forms are generally not amenable to dose titrations of this nature.

A few modified release liquids have been developed to overcome the limitations associated with solid dosage forms. Typically, these compositions are aqueous suspensions or emulsions of coated particles containing active agent or active agent absorbed on a carrier, such as a sugar or other water-soluble core. Other materials, such as ion-exchange resins, can also be used as the carrier.

Compositions containing an ion-exchange resin as a carrier, which are suitable for oral administration, are typically aqueous suspensions and are essentially the only form described in the art.

U.S. Pat. Nos. 4,221,778 and 4,847,077 to Raghunathan describes prolonged release pharmaceutical compositions containing ion-exchange resin drug complexes at least a substantial portion of which have been treated with a solvating (impregnating) agent and coated with a diffusion barrier coating. Pre-treatment of the resin drug complex with a solvating agent is necessary in order to coat the particles with a diffusion barrier coating.

U.S. Pat. No. 4,894,239 to Nonomura et al. describes a sustained release microcapsule formulation containing an ion-exchange resin with 6 to 16% crosslinking, containing a drug absorbed in an amount not less than 80% of its theoretical ion absorption amount and coated with a water permeable polymer. The microcapsules are suspended in water for oral administration.

U.S. Pat. No. 4,996,047 to Kelleher et al. describes oral pharmaceutical preparations which comprise a pharmacologically active drug bound to small particles of an ion-exchange resin to provide a drug-resin complex having a drug content above a specified value. The drug-resin complex is coated with a water-permeable diffusion barrier coating that is insoluble in gastrointestinal fluids. The drug-resin particles are suitable for suspension in an essentially aqueous vehicle. Liquid oral dosage forms are prepared by dissolving or dispersing the drug-resin particles in an aqueous pharmaceutical carrier.

U.S. Pat. No. 5,071,646 to Malkowska et al. describes an ion-exchange resin composition, which is dispersible in water. The resin composition comprises a granulated ion-exchange resin, a pharmacologically active ingredient bound thereto with a sugar or sugar alcohol. The composition can be administered in a capsule or sachet.

Compositions containing drug-loaded ion-exchange resin particles have also been developed for topical administration.

U.S. Pat. No. 4,692,462 to Banerjee describes pharmaceutical compositions containing a pharmacologically active drug in combination with a non-toxic pharmaceutically acceptable ion-exchange resin and a salt in a gel-forming vehicle which is suitable for topical administration. The drug-resin complex and the desired salt, and a penetrating agent, if desired, are mixed with deionized water containing a gel-forming polymer. The gel matrix is then poured in a cavity of inert material to form the transdermal device (i.e. a patch).

U.S. Pat. No. 5,296,228 to Chang et al. describes sustained release pharmaceutical compositions containing drug-loaded ion-exchange resin particles incorporated in an aqueous reversibly gelling polymeric solution. The compositions are aqueous solutions, which can be administered by injection or as drop instillable liquids or liquid sprays.

U.S. Pat. No. 5,275,820 to Chang describes sustained release pharmaceutical compositions containing drug-loaded ion-exchange resin particles incorporated into an erodible polymeric matrix or microcapsule to form microparticulates. The microparticulates are suspended in a liquid carrier where the encapsulating polymeric matrix shields the drug-loaded ion-exchange resin from solvent interactions. Preferred liquid carriers include de-ionized water and substantially non-ionic water. The compositions are liquid suspensions, which can be administered by injection or as drop instillable liquids or liquid sprays.

U.S. Pat. No. 5,368,852 to Umemoto et al. describes prolonged-release liquid pharmaceutical preparations prepared by coating a pharmaceutically active drug-ion exchange resin complex, which was treated previously with an impregnating agent, with a water permeable diffusion barrier material, followed by suspending the coated complex in a solution containing preservatives. The solvent used to prepare the liquid formulations may be an aqueous solvent or an oil solvent. The compositions can be formulated for oral administration, nasal administration or as an ophthalmic solution.

Emulsions and oils may be used to dissolve hydrophobic materials, such as prostaglandins (see U.S. Pat. No. 3,903,297), and antibiotics (see U.S. Pat. No. 5,260,292), but are not used to prepare suspensions of active agent loaded ion exchange resins in a non-ionic, non-aqueous vehicle. Likewise, intramuscular injectable materials may contain active agents in a lipid vehicle (e.g. DepoProvera), and implants or temporary implants may be essentially non-aqueous (see. U.S. Pat. No. 4,931,279), but in neither case is the active agent-containing material designed to vanish after a short period.

There is a need for stabilized active agent preparations, preferably with delayed or controlled release features, that can be formulated into final dosage forms that are easier to swallow, provide controlled-release benefits in topical applications, or offer more versatile options for flexible dosing than previously available.

It is therefore an object of the present invention to provide easy to administer active agent formulations that provide modified release of one or more active agents.

It is a further object of the invention to provide non-oral routes for administration of active agents via carrier complexes, particularly by topical or intracavity administration.

It is a further object of the invention to provide vehicles for oral or topical delivery of controlled release active agent forms to patients, by using non-ionic non-aqueous (“NINA”) liquids, emulsions, semisolids and soft solids as delivery vehicles.

### SUMMARY OF THE INVENTION

An improved controlled release composition for non-parenteral administration of one or more active agents, particularly for oral or topical administration, has been developed. The formulation is prepared by dissolving or dispersing one or more active agents bound to a carrier in one or more non-ionic, non-aqueous (“NINA”) vehicles. The active agent/carrier complex is typically in the form of small, porous or high-surface-area particles, which are less than about 150 microns in diameter, such as ion-exchange resin particles. The active agent/carrier complex can be coated with one or more coating materials to modify the release of the active agent.

The NINA vehicle can control the rate of efflux of active agent from the carrier, as well as the rate of permeation of water to the active agent/carrier complex. Therefore, active agent release can be controlled, especially in topical applications, by varying the hydrophilicity and/or viscosity of the NINA vehicle. Selection of the appropriate excipients, such as suspension agents and stabilizing agents can also be used to modify the release of the active agent.

The NINA vehicle can also serve as a solvent for one or more active agents. The NINA vehicle allows for the incorporation of both water-soluble active agents and lipid-soluble active agents in the same dosage form, such as a soft gelatin capsule. The combination of disparate active agents in a single dosage form can result in decreased costs and increased patient compliance.

Replacing traditional aqueous vehicles with one or more NINA vehicles, such as an oil or an ointment, allows the active agent-loaded ion exchange particles, with or without coatings, to be administered either orally or topically, or both. Moreover, the NINA vehicle can also allow the active agents to be stored in a vehicle, ready for administration, in a substantially anhydrous environment. This can lead to markedly increased stability for some active agents.

### BRIEF DESCRIPTION OF THE FIGURES

FIG. 1 shows the release profile of albuterol from a resinate suspended in different NINA vehicles compared to the direct release of the drug into aqueous buffer.

### DETAILED DESCRIPTION OF THE INVENTION

### DEFINITIONS

Modified release dosage form: A modified release dosage form is one for which the active agent release characteristics of time, course, and/or location are chosen to accomplish therapeutic or convenience objectives not offered by conventional dosage forms such as solutions, conventional ointments, or promptly dissolving dosage forms. Delayed release, extended release, and pulsatile release dosage forms and their combinations are examples of modified release dosage forms. Modified release kinetics can also be obtained by controlling the rate of uptake of water and/or ions by a vehicle containing one or more active agents bound to a carrier.

Delayed release dosage form: A delayed release dosage form is one that releases an active agent (or active agents) at a time other than promptly after administration.

Extended release dosage form: An extended release dosage form is one that allows at least a twofold reduction in dosing frequency as compared to that active agent presented as a conventional dosage form (e.g. as a solution or prompt active agent-releasing, conventional solid dosage form).

Pulsatile release dosage form: A pulsatile release dosage form is one that mimics a multiple dosing profile without repeated administration and allows at least a twofold reduction in dosing frequency as compared to that active agent presented as a conventional dosage form (e.g. as a solution or prompt active agent-releasing, conventional solid dosage form). For example, a pulsatile formulation could contain equal amounts of immediate release particles and of delayed release coated particles.

As used herein the term “taste masking coating” refers to a pH dependent coating that is insoluble in the mouth but dissolves in the acidic pH of the stomach.

As used herein the term “extended release coating” refers to a pH independent substance that will act as a barrier to control the diffusion of the active agent from its core complex into the gastrointestinal fluids.

As used herein, the term “enteric coating” refers to a coating material which remains substantially intact in the acid environment of the stomach, but which dissolves in the environment of the intestines.

As used herein the term “delayed release coating” refers to a pH dependent coating that is insoluble in the acidic pH of the stomach, the pH within the upper small intestine, but dissolves within the lower small intestine or upper large intestine.

As used herein, the term “water permeation control coating” refers to a coating on an active agent/carrier complex used for topical application, wherein the coating controls the rate at which water, from sources such as sweat, wound exudate and/or atmospheric moisture, enters the active agent/carrier complex, or the rate at which the active agent releases from the carrier.

The term “non-aqueous” refers to a vehicle for delivery of the active agent/carrier complex that is substantially free of water. A non-aqueous vehicle may further be rendered anhydrous if required for stability of the active agent or the formulation.

The term “NINA” (an acronym for “non-ionic non-aqueous”) is used herein to designate a non-ionic, substantially non-aqueous, liquid, semi-solid or soft solid material used as a vehicle for delivery of active agent/carrier complexes or resinates (i.e., active agents bound or adsorbed to a “carrier”), from any source, including animal, vegetable, mineral and synthetic. NINA vehicles are selected to be compatible with the skin for topical administration, and compatible with the gastrointestinal tract for oral administration.

As used herein, a “bandage” is a porous solid macroscopic carrier that does not dissolve in water or in the NINA, but which will allow permeation of aqueous liquids to the loaded resin after application. “Mucoadhesive” refers to compounds that adhere to mucosal surfaces, including, but not limited to, polycarboxylic acid materials and polyanhydrides. “Topical” administration as used herein includes not only administration to the skin, but also includes direct application to accessible body cavities, including the mouth, the nose, the ears, the eyes, the urethra, the vagina and the rectum. Topical is distinguished from “oral” administration, which refers to administration to the gastrointestinal tract via the mouth.

As used herein, “carrier” refers to a particulate material which can complex one or more active agents. A preferred class of carrier is a “resin”, which includes polymeric materials used as carriers acting via ion exchange, absorption, etc. The term resin is sometimes used more broadly herein, unless otherwise distinguished, to include other particulate materials useable as carriers, including, but not limited to, charged inorganic materials.

As used herein, “complex” refers to covalent, ionic, hydrophobic and polar interactions. Examples of polar interactions include hydrogen bonding. Examples of hydrophobic interaction include Van der Waals forces, pi stacking, etc.

I. Active Agent/Carrier Complexes

The carrier-bound active agent compositions described herein demonstrate several types of release profiles. The carrier-bound active agent compositions are obtained by complexing one or more active agents with one or more pharmaceutically acceptable binding resins or other carriers. The complexes are dissolved or dispersed in one or more non-ionic, non-aqueous vehicles. The complexes can be coated with one or more coatings to modify the release of the active agent from the complex.

A. Active agents to be Formulated

Exemplary active agents useful for forming the composition described herein include, but are not limited to, analeptic agents; analgesic agents; anesthetic agents; antiasthmatic agents; antiarthritic agents; anticancer agents; anticholinergic agents; anticonvulsant agents; antidepressant agents; antidiabetic agents; antidiarrheal agents; antiemetic agents; antihelminthic agents; antihistamines; antihyperlipidemic agents; antihypertensive agents; anti-infective agents; anti-inflammatory agents; antimigraine agents; antineoplastic agents; antiparkinsonism active agents; antipruritic agents; antipsychotic agents; antipyretic agents; antispasmodic agents; antitubercular agents; antiulcer agents; antiviral agents; anxiolytic agents; appetite suppressants (anorexic agents); attention deficit disorder and attention deficit hyperactivity disorder active agents; cardiovascular agents including calcium channel blockers, antianginal agents, central nervous system (“CNS”) agents, beta-blockers and antiarrhythmic agents; central nervous system stimulants; diuretics; genetic materials; hormonolytics; hypnotics; hypoglycemic agents; immunosuppressive agents; muscle relaxants; narcotic antagonists; nicotine; nutritional agents; parasympatholytics; peptide active agents; psychostimulants; sedatives; sialagogues, steroids; smoking cessation agents; sympathomimetics; tranquilizers; vasodilators; beta-agonist; and tocolytic agents.

The active agent is selected based on inclusion in the molecule of a group, such as an amino group, which will readily bind to a charged complexing agent such as an ion-exchange resin. Any active agent that bears an acidic or a basic functional group, for example, an amine, imine, imidazoyl, guanidine, piperidinyl, pyridinyl, quaternary anunonium, or other basic group, or a carboxylic, phosphoric, phenolic, sulfuric, sulfonic or other acidic group, can be bound to a resin of the opposite charge. Representative active agent agents are described in, for example, WO 98/18610 by Van Lengerich; U.S. Pat. No. 6,512,950 to Li et al. and U.S. Pat. No. 4,996,047 to Kelleher et al.

Examples of active agents that bear acidic or basic functional groups and thus may be complexed with a binding resin include, but are not limited to Acetylsalicylic acid, Alendronic acid, Alosetron, Amantadine, Amlopidine, Anagrelide, Argatroban, Atomoxetine, Atrovastatin, Azithromycin dehydrate, Balsalazide, Bromocriptan, Bupropion, Candesartan, Carboplatin, Ceftriaxone, Clavulonic acid, Clindamycin, Cimetadine, Dehydrocholic (acid), Dexmethylphenidate, Diclofenac, Dicyclomine, Diflunisal, Diltiazem, Donepezil, Doxorubicin, Doxepin, Epirubicin, Etodolic acid, Ethacrynic acid, Fenoprofen, Fluoxetine, Furosemide, Gemfibrozil, Hydroxyzine, Ibuprofen, Imipramine, Levothyroxine, Maprolitline, Meclizine, Methadone, Methylphenidate, Minocycline, Mitoxantone, Moxifloxacin, Mycophenolic acid, Naproxen, Niflumic acid, Ofloxacin, Ondansetron, Pantoprazole, Paroxetine, Pergolide, Pramipexole, Phenytoin, Pravastain, Probenecid, Rabeprazole, Risedronic acid, Retinoic acid, Ropinirole, Selegiline, Sulindac, Tamsulosin, Telmisertan, Terbinafine, Theophyline, Tiludronic Acid, Tinzaparin, Ticarcillin, Valproic acid, Salicylic acid, Sevelamer, Ziprasidone, Zoledronic acid, Acetophenazine, Albuterol, Almotriptan, Amitriptyline, Amphetamine, Atracurium, Beclomethasone, Benztropine, Biperiden, Bosentan, Bromodiphenhydramine, Brompheniramine carbinoxamine, Caffeine, Capecitabine, Carbergoline, Cetirizine, Chlocylizine, Chlorpheniramine, Chlorphenoxamine, Chlorpromazine, Citalopram, Clavunate potassium, Ciprofloxacin, Clemastine, Clomiphene, Clonidine, Clopidogrel, Codeine, Cyclizine, Cyclobenzaprine, Cyproheptadine, Delavirdine, Diethylpropion, Divalproex, Desipramine, Dexmethylphenidate, Dexbrompheniramine, Dexchlopheniramine, Dexchlor, Dextroamphetamine, Dexedrine, Dextromethorphan, Diphemanil methylsulphate, Diphenhydramine, Dolasetron, Doxylamine, Enoxaparin, Ergotamine, Ertepenem, Eprosartan, Escitalopram, Esomeprazole, Fenoldopam, Fentanyl, Fexofenadine, Fluvastatin, Fluphenazine, Fluticasone, Fosinopril, Frovatriptan, Gabapentin, Galatamine, Gatifloxacin, Gemcitabine, Haloperidol, Hyalurondate, Hydrocodone, Hydroxychloroquine, Hyoscyamine, Imatinib, Imipenem, Ipatropin, Lisinopril, Leuprolide, Levopropoxyphene, Losartan, Mesalamine, Mepenzolate, Meperidine, Mephentermine, Mesalimine, Mesoridazine, Metaproteranol, Metformin, Methdialazine, Methscopolamine, Methysergide, Metoprolol, Metronidazole, Mibefradil, Montelukast, Morphine, Mometasone, Naratriptan, Nelfinavir, Nortriptylene, Noscapine, Nylindrin, Orphenadrine, Oseltamivir, Oxybutynin, Papaverine, Pentazocine, Phendimetrazine, Phentermine, Pioglitazone, Pilocarpine, Prochloroperazine, Pyrilamine, Quetapine, Ranitidine, Rivastigmine, Rosiglitazone, Salmetrol, Sertaline, Sotalol, Sumatriptan, Tazobactam, Tacrolimus, Tamoxifen, Ticlopidine, Topiramate, Tolterodine, Triptorelin, Triplennamine, Triprolidine, Tramadol, Trovofloxacin, Ursodiol, Promazine, Propoxyphene, Propanolol, Pseudoephedrine, Pyrilamine, Quinidine, Oxybate sodium, Sermorelin, Tacrolimus, Tegaseroid, Teriparatide, Tolterodine, Triptorelin pamoate, Scoplolamine, Venlafaxine, Zamivir, Aminocaproic acid, Aminosalicylic acid, Hydromorphone, Isosuprine, Levorphanol, Melhalan, Nalidixic acid, and Para-aminosalicylic acid.

Pharmaceutically acceptable salts of the above compounds may also be used.

B. Carriers

Active agent/carrier complexes are generally prepared by complexing the active agent with a pharmaceutically acceptable carrier. The complex can be formed by reaction of a functional group on the active agent with a functional group on the carrier. Alternatively, the complex can be formed by the overall interaction of the active agent and the carrier, for example, via hydrophobic forces (Van Der Waals forces, pi stacking, etc.) or hydrogen bonding, or by entrapping the active agent within or on the carrier, for example following drying of an applied solution.

Suitable carriers include, but are not limited to, ion exchange resins; charged absorbents other than polymeric resins, including charged inorganic particulates such as silicates, aluminosilicates, and other inorganic particulates as well as particulate or crosslinked forms of natural polymers. Examples of derivatized natural polymer resins include but are not limited to, carboxymethyl cellulose, particulate forms of chitin, chitosan, and partially deacetylated chitin. Crosslinked forms of polymers such as glucomanans, galactomannans, galactoaminogylcans, glycosaminoglycans, hyaluronic acid, chondroitin sulfate, or polylysine can also be used as carriers.

In one embodiment, the binding resin is an ion exchange resin. For example, an active agent having a basic group such as an amino group can complex with an ion-exchange resin that bears an acidic group such as a sulfate or carboxylate group. Conversely, an active agent that has an acidic group can complex with an ion-exchange resin that bears a basic group. Active agents administered orally are released by exchanging with appropriately charged ions within the gastrointestinal tract. Active agents applied topically are released by fluids present on the skin, such as sweat, atmospheric moisture, or wound exudate, which either contain ions, or can liberate ions, when required, for release of the active agent from the carrier, from the skin or from separate ionic depots within the NINA vehicle.

Ion-exchange resins are water-insoluble materials, often cross-linked polymers, containing covalently bound salt forming groups in repeating positions on the polymer chain. The ion-exchange resins suitable for use in these preparations consist of a pharmacologically inert organic or -inorganic matrix. The organic matrix may be synthetic (e.g., polymers or copolymers of acrylic acid, methacrylic acid, sulfonated styrene, sulfonated divinylbenzene), or partially synthetic (e.g., modified cellulose and dextrans). The ion exchange carrier can also be inorganic, e.g., silica gel, or aluminosilicates, natively charged or modified by the addition of ionic groups.

The covalently bound salt forming groups may be strongly acidic (e.g., phosphoric, sulfonic or sulfuric acid groups), weakly acidic (e.g., carboxylic acid), strongly basic (e.g., quaternary ammonium), weakly basic (e.g., primary amine), or a combination of these types of groups. Other types of charged groups can also be used, including any organic moiety that bears an acidic or a basic group, for example, an amine, imine, imidazoyl, guanidine, pyridinyl, quaternary ammonium, or other basic group, or a carboxylic, phosphoric, phenolic, sulfuric, sulfonic, boric, boronic, or other acidic group.

In general, those types of ion-exchangers suitable for use in ion-exchange chromatography and for such applications as deionization of water are suitable for use in the controlled release compositions described herein. Such ion-exchangers are described by H. F. Walton in “Principles of Ion Exchange” (pp. 312-343) and “Techniques and Applications of Ion-Exchange Chromatography” (pp. 344-361) in Chromatography. (E. Heftmann, editor), Van Nostrand Reinhold Company, New York (1975). The organic ion-exchange resins typically have exchange capacities below about 6 meq./g (i.e., 1 ionic group per 166 daltons of resin) and more commonly below about 5.5 meq./g.

Suitable ion-exchange resins include, but are not limited to commercially available ion exchange resins such as Dowex® and other resins available from Dow Chemical; Amberlite® and Amberlyst® and other resins available from Rohm and Haas; Indion® resins available from Ion Exchange, Ltd. (India), Diaion® resins by Mitsubishi; BioRex Type AG and other resins available from BioRad; Sephadex® and Sepharose® available from Amersham; resins by Lewatit, available from Fluka; Toyopearl® resins available from Toyo Soda; IONAC® and Whatman resins available from VWR; and BakerBond® resins available from J T Baker.

Preferred ion exchange resins will be those supplied in grades known to be suitable for delivery of pharmaceuticals. Particular resins believed to be useful and approved include, without limitation, Amberlite® IRP-69 (Rohm and Haas), and INDION® 224, INDION® 244, and INDION® 254 (Ion Exchange (India) Ltd.). These resins are sulfonated polymers composed of polystyrene cross-linked with divinylbenzene.

The size of the ion-exchange particles is less than about 2 millimeters, preferably less than about 1000 microns, more preferably less than about 500 microns, most preferably less than about 150 micron (about 40 standard mesh). Commercially available ion-exchange resins (including Amberlite IRP-69, INDION 244 and INDION 254 and numerous other products) are typically available in several particle size ranges, and many have an available particle size range less than 150 microns. The particle size is not usually a critical variable in terms of active agent release rate, but large particles can give a formulation a “gritty” feel, which is not desirable. When a formulation is a spray or an aerosol, the preferred particle size is less than about 100 microns, preferably less than about 50 microns, and more preferably less than about 20 microns. Particle size can be reduced before use, preferably before active agent loading, by milling, grinding and other known particle size-reduction techniques

As used herein, the term “regularly shaped particles” refer to those particles which substantially conform to geometric shapes such as spherical, elliptical, and cylindrical. As used herein, the term “irregularly shaped particles” refers to particles excluded from the above definition, such as those particles with amorphous shapes with increased surface areas due to channels or distortions, or subsequent to grinding. For example, irregularly shaped ion-exchange resins of this type are exemplified by Amberlite IRP-69 (supplied by Rohm and Haas), and to the active agent-resin complexes formed by binding active agents to these resins. Irregularly or regularly shaped particles may be used. The distinction between regularly shaped and irregularly shaped particles has been found by Kelleher et al (U.S. Pat. No. 4,996,047) to affect the degree of active agent loading required to prevent swelling and rupture of coating when loaded resins are placed in salt solutions, in the absence of fillers or impregnating agents, such as polyethylene glycol. They found that the critical value was at least 38% active agent (by weight in the active agent/resin complex) in irregular resins, and at least 30% by weight in regular resins.

Ion exchange resins have pores of various sizes, which expand the area available for active agent binding. The typical pore diameter is in the range of about 30 to 300 nanometers (nm), which is large enough for access by small-molecule active agents. For large active agents, such as proteins or nucleic acids, resins with larger pores, such as 500 to 2000 nm (0.5 to 2 micron), often called “macroreticular” or “macroporous”, are preferred.

Binding of active agent to a charged (ion-exchange) resin can be accomplished according to any of four general reactions. In the case of a basic active agent, these are: (a) resin (Na-form) plus active agent (salt form); (b) resin (Na-form) plus active agent (as free base); (c) resin (H-form) plus active agent (salt form); and (d) resin (H-form) plus active agent (as free base). Other pharmaceutically acceptable cations, especially K and Li, can be substituted for Na. All of these reactions except (d) have cationic by-products and these by-products, by competing with the cationic active agent for binding sites on the resin, reduce the amount of active agent bound at equilibrium. For basic active agents, stoichiometric binding of active agent to resin, i.e., binding an applied active agent molecule to essentially each binding site while having a very low level of active agent left in solution, is accomplished only through reaction (d).

Four analogous binding reactions can be carried out for binding an acidic active agent to an anionic exchange resin. These are: (a) resin (Cl-form) plus active agent (salt form); (b) resin (Cl-form) plus active agent (as free acid); (c) resin (as free base) plus active agent (salt form); and (d) resin (as free base) plus active agent (as free acid). Other pharmaceutically acceptable anions, especially Br, acetate, lactate and sulfate, can be substituted for Cl. All of these reactions except (d) have ionic by-products and the anions generated when the reactions occur compete with the anionic active agent for binding sites on the resin with the result that reduced levels of active agent are bound at equilibrium. For acidic active agents, stoichiometric binding of active agent to resin (as above) is accomplished only through reaction (d).

Active agent is bound to the resin by exposure of the resin to the active agent in solution via a batch process or a continuous process (such as in a chromatographic column). The active agent-resin complex thus formed is collected by filtration and washed with an appropriate solvent to insure removal of any unbound active agent or by-products. The complexes are usually air-dried in trays. Such processes are described in, for example, U.S. Pat. No. 4,221,778 to Raghunathan; U.S. Pat. No. 4,894,239 to Nonomura et al.; and U.S. Pat. No. 4,996,047 to Kelleher et al. Similar processes can also be used with ionic carriers other than ion exchange resins, such as silicates and other inorganic particles. However, these complexes may require collection by centrifugation or ultrafine filtration because of their small particle size.

The result of treating the ion exchange resin with a solution of active agent is an active agent-loaded particle with no coating. Such a particle can be used for active agent delivery with no additional treatment, especially in topical formulations. However, the loaded particles will typically be coated with one or more layers of materials to control the rate and location of release of active agent from the resin when the particles come in contact with a salt-containing aqueous solution, such as saliva, gastric juice or sweat.

C. Coatings

i. Taste Masking Coatings

Although binding active agent to ion-exchange resins is a method of taste-masking known in the pharmaceutical art, some unpleasant taste may be experienced when uncoated active agent-resin complexes are placed in the mouth. This may be a consequence of ion-exchange that occurs during the time that the active agent-resin complexes are in the mouth, and may be a particular problem for chewable or rapidly dissolving solid formulations. Release of a bitter compound within the mouth makes such active agent loaded ion-exchange resin particles or other carriers unpalatable and irritating to the throat and esophagus.

The active agent-carrier particles can be coated with a taste masking coating. The taste masking coating prevents the release of active agent within the mouth and insures that no unpleasant, bitter flavor is experienced by the patient consuming the dosage form.

Suitable taste masking coatings include the cationic polymer Eudragit® E 100 (Rohm Pharma), which contains amino groups. Such films are insoluble in the neutral medium of saliva, but dissolve in the acid environment of the stomach. Film coatings with a thickness of approximately 10 micrometers can prevent medication with a bitter or unpleasant taste from dissolving in the mouth upon ingestion or during swallowing. The protective film dissolves quickly in the stomach allowing for the active ingredient to be released. A sugar coating may be used to accomplish a similar taste-masking effect, but such a coating must be much thicker than the polymeric coating, and the enlarged particles may result in tickling or irritation of the throat.

ii. Enteric Coatings

In some embodiments, the active agent-carrier complexes are coated with a pH sensitive polymer which is insoluble in the acid environment of the stomach, and soluble in the more basic environment of the GI tract. This is known as an enteric coating, because it creates a dosage form designed to prevent active agent release in the stomach. Preventing active agent release in the stomach has the advantage of reducing side effects associated with irritation of the gastric mucosa, and of minimizing exposure of active agent to very low pH, which can result in degradation of the active agent. Avoiding release within the stomach can be achieved using enteric coatings known in the art. The enteric coated formulation remains intact or substantially intact in the stomach; however, once the formulation reaches the small intestines, the enteric coating dissolves and exposes either active agent-containing carrier particles or active agent-containing carrier particles coated with extended release coating to the surrounding environment.

The enteric coated particles can be prepared as described in references such as “Pharmaceutical dosage form tablets”, eds. Liberman et. al. (New York, Marcel Dekker, Inc., 1989), “Remington—The science and practice of pharmacy”, 20th ed., Lippincott Williams & Wilkins, Baltimore, Md., 2000, and “Pharmaceutical dosage forms and active agent delivery systems”, 6th Edition, Ansel et. al., (Media, P A: Williams and Wilkins, 1995). Examples of suitable coating materials include but are not limited to cellulose polymers, such as cellulose acetate phthalate, hydroxypropyl cellulose, hydroxypropyl methylcellulose phthalate and hydroxypropyl methylcellulose acetate succinate; polyvinyl acetate phthalate, acrylic acid polymers and copolymers, and certain methacrylic resins that are commercially available under the trade name Eudragit® (Rohm Pharma).

Additionally the coating material may contain conventional carriers such as plasticizers, pigments, colorants, glidants, stabilization agents, and surfactants.

iii. Extended Release Coatings

Extended release pharmaceutical compositions are obtained by complexing active agent with pharmaceutically acceptable carrier particles, and coating such complexes with a substance that will act as a barrier to control the diffusion of the active agent from its core complex into the gastrointestinal fluids.

Control of the release of active agents from active agent-carrier complexes is possible with the use of a diffusion barrier coating on the active agent-carrier complex particles. Several processing methods to apply extended release coatings on active agent loaded carrier particles have been described, see for example, U.S. Pat. Nos. 4,996,047, 4,221,778, and 4,894,239. Any of these may be used to obtain the extended release active agent composition.

In general, any coating procedure which provides a contiguous coating on each particle of active agent-carrier complex without significant agglomeration of particles may be used. Coating procedures known in the pharmaceutical art including, but not limited to, fluid bed coating processes and microencapsulation, may be used to obtain appropriate coatings. The coating materials may be any of a large number of natural or synthetic film-formers used singly, in admixture with each other, and in admixture with plasticizers (for example, Durkex 500 vegetable oil), pigments and other substances to alter the characteristics of the coating.

Typically, the major components of the coating are insoluble in, and permeable to, water. However, with non-aqueous NINA vehicles, it may be desirable to use a water-soluble substance, such as methyl cellulose, or a sugar, alone or with other materials in forming the coating. The coating materials may be applied as a suspension in a non-ionic aqueous fluid or a non-aqueous fluid, or as a solution in organic solvents. A water-permeable diffusion barrier may comprise ethyl cellulose, methyl cellulose and mixtures thereof. The water-permeable diffusion barrier may also comprise water insoluble synthetic polymers sold under the trade name Eudragit® (Rohm Pharma), such as Eudragit R S, Eudragit R L, Eudragit N E and mixtures thereof. Other examples of such coating materials can be found in the Handbook of Pharmaceutical Excipients, Ed. By A. Wade and P. J. Weller, (1994).

As used herein, the term water-permeable is used to indicate that the fluids of the alimentary canal or those found on the skin will permeate or penetrate the coating film. The fluids may or may not dissolve the film, in whole or in part. Depending on the permeability or solubility of the coating (polymer or polymer mixture) a lighter or heavier application of the coating is required to obtain the desired release rate. Moreover, because of the NINA vehicle, sugars and other water-soluble materials may be used as a coating, using application techniques well known in the art.

In addition to the known methods of processing active agent-loaded carriers to obtain stable extended release coatings, it was found that the coating of active agent loaded ion-exchange carriers with an acrylic polymer based coating, such as Eudragit RS, results in a stable extended release composition without the need for impregnating agents. This is true even when the active agent loading is conducted by binding the salt form of the active agent with the salt form of the carrier, rather than binding the free base of the active agent with carrier in its acidic form as described in U.S. Pat. Nos. 4,996,047 to Kelleher, et al. and U.S. Pat. No. 4,894,239 to Nonomura et al. Stable extended release coatings with out the need for impregnating agents have been prepared with active-agent loadings lower than those reported by Kelleher and Nonomura. However, a proposed polymer coating should be tested with the particular NINA vehicle, to insure that the active agent is not eluted from the carrier during storage in the NINA vehicle.

iv. Delayed Release Coatings

In some embodiments active agent-carrier complexes are coated with a pH sensitive polymer which is insoluble in the acid environment of the stomach, insoluble in the environment of the small intestines, and soluble in the conditions within the lower small intestine or upper large intestine (e.g., above pH 7.0). Such a delayed release form is designed to prevent active agent release in the upper part of the gastrointestinal (GI) tract.

The delayed release particles can be prepared by coating active agent-containing carrier microparticles with a selected coating material. Preferred coating materials are comprised of bioerodible, gradually hydrolyzable, gradually water-soluble, and/or enzymatically degradable polymers, and may be conventional “enteric” polymers. Enteric polymers, as will be appreciated by those skilled in the art, become soluble in the higher pH environment of the lower gastrointestinal tract or slowly erode as the dosage form passes through the gastrointestinal tract, while enzymatically degradable polymers are degraded by bacterial enzymes present in the lower gastrointestinal tract, particularly in the colon. Suitable coating materials for effecting delayed release include, but are not limited to, cellulosic polymers such as hydroxypropyl cellulose, hydroxyethyl cellulose, hydroxymethyl cellulose, hydroxypropyl methyl cellulose, hydroxypropyl methyl cellulose acetate succinate, hydroxypropylmethyl cellulose phthalate, methylcellulose, ethyl cellulose, cellulose acetate, cellulose acetate phthalate, cellulose acetate trimellitate and carboxymethylcellulose sodium; acrylic acid polymers and copolymers, preferably formed from acrylic acid, methacrylic acid, methyl acrylate, ethyl acrylate, methyl methacrylate and/or ethyl methacrylate, and other methacrylic resins that are commercially available under the tradename Eudragit®. (Rohm Pharma; Westerstadt, Germany), including Eudragit® L30D-55 and L100-55 (soluble at pH 5.5 and above), Eudragit® L-100 (soluble at pH 6.0 and above), Eudragit®. S (soluble at pH.7.0 and above, as a result of a higher degree of esterification), and Eudragit®, NE, RL and RS (water-insoluble polymers having different degrees of permeability and expandability). Additional polymers include vinyl polymers and copolymers such as polyvinyl pyrrolidone, vinyl acetate, vinylacetate phthalate, vinylacetate crotonic acid copolymer, and ethylene-vinyl acetate copolymer; enzymatically degradable polymers such as azo polymers, pectin, chitosan, amylose and guar gum; and shellac. Combinations of different coating materials may also be used. Multi-layer coatings using different polymers may also be applied.

v. Water Permeation Control Coatings

As noted above, the types of coatings used to control the rate of release of the active agent in oral formulations, can also be used to control the rate of release of the active agent in topical formulations. Some experimentation to determine what sort of coating is best may be required, since the skin, and even wounds, are generally drier than any region of the GI tract. Water permeation control coatings may control the rate at which water, from sources, such as sweat, wound exudates, urine, and/or atmospheric moisture, enters the active agent carrier complex or the rate at which the active agent releases from the carrier.

vi. Other Coating Considerations

The preferred coating weights for particular coating materials may be readily determined by those skilled in the art by evaluating individual release profiles for active agent loaded ion exchange resins with different quantities of various coating materials. The coating should be tested for compatibility with the selected NINA vehicle. If there is to be an outer coating of a taste-masking material or other type of material which is stable in the NINA vehicle, then less NINA stability of an inner coating is required. Hence, an inner coating, such as an enteric coating or extended release coating, can be protected from the vehicle by an outer taste-masking coating or a water-soluble coating that is resistant to a NINA vehicle but not to water, and therefore traditional enteric coatings, extended release coatings, and delayed release coating known for use in aqueous environments will in many cases be suitable for use in a NINA vehicle as well.

The coating composition may include conventional additives, such as plasticizers, pigments, colorants, stabilizing agents, glidants, etc. A plasticizer is normally present to reduce the fragility of the coating, and will generally represent about 10 wt. % to 50 wt. % relative to the dry weight of the polymer. Examples of typical plasticizers are, but not limited to, polyethylene glycol, propylene glycol, triacetin, dimethyl. phthalate, diethyl phthalate, dibutyl phthalate, dibutyl sebacate, triethyl citrate, tributyl citrate, triethyl acetyl citrate, castor oil and acetylated monoglycerides. Note that many materials used as plasticizers are also candidates for NINA vehicles.

A stabilizing agent is preferably used to stabilize particles in the dispersion. Typical stabilizing agents are nonionic emulsifiers such as sorbitan esters, polysorbates and polyvinylpyrrolidone.

Glidants are recommended to reduce sticking effects during film formation and drying, and will generally represent approximately 25 wt. % to 100 wt. % of the polymer weight in the coating solution. One effective glidant is talc. Other glidants such as magnesium stearate and glycerol monostearates may also be used. Pigments such as titanium dioxide may also be used. Small quantities of an anti-foaming agent, such as a silicone (e.g., simethicone), may also be added to the coating composition.

Delayed release coated particles can be administered simultaneously with an immediate release dose of the active agent. Such a combination produces the modified release profile referred to as “pulsatile release”. By “pulsatile” is meant that active agent doses are released at spaced apart intervals of time. Generally, upon ingestion of the dosage form, release of the initial dose is substantially immediate, i.e., the first active agent release “pulse” occurs within about one hour of ingestion. This initial pulse is followed by a first time interval (lag time) during which very little or no active agent is released from the dosage form, after which a second dose is then released. Optionally, a second pulse is followed by a second time interval (lag time) during which very little or no active agent is released from the dosage form, after which a third dose is then released.

The first pulse of the pulsatile release composition can be obtained by administering unmodified active agent, uncoated active agent-carrier particles, taste-masked coated active agent-carrier particles, or, in some cases, enteric coated active agent-carrier-particles along with delayed release coated particles that provide a second pulse.

In some cases it may be advantageous to combine an immediately releasing dose of active agent (e.g., unmodified active agent, uncoated active agent-carrier particles, or taste masking coated active agent-carrier particles) with enteric coated active agent-carrier particles to create a pulsatile profile. In this case the first pulse will occur substantially immediately after administration and the second pulse will occur once the enteric coating has dissolved (in the upper small intestines).

In order to create a final dosage form with three pulses, an immediate release dose of active agent (e.g., unmodified active agent, uncoated active agent-carrier particles, or taste masking coated active agent-carrier particles) can be combined with enteric coated active agent-carrier particles and delayed release coated active agent carrier particles.

In some cases where receptors are subject to saturation with a given active agent, a distinct drop in plasma concentration may be required for optimal therapeutic performance. In these cases separating the first and second pulse of release by a significant time lag may be critical and may require the use of delayed release coated particles (rather than conventional enteric coated particles) in combination with an immediate release dose.

One of the advantages of a delayed release formulation may be diminished incidence or reduced intensity of active agent side effects, when compared to an immediate release form. A very common side effect that can be prevented is nausea. Other preventable side effects include vomiting, headache, tremulousness, anxiety, panic attacks, palpitations, urinary retention, orthostatic hypotension, diaphoresis, chest pain, rash, weight gain, back pain, constipation, vertigo, increased sweating, agitation, hot flushes, tremors, fatigue, somnolence, dyspepsia, dysoria, nervousness, dry mouth, abdominal pain, irritability, and insomnia.

II. Formulations Containing Carrier-Bound Active Agent Compositions

A. Non-Ionic, Non-Aqueous Vehicles

Formulations are prepared using a pharmaceutically acceptable non-ionic, non-aqueous vehicle composed of materials that are considered safe and effective and may be administered to an individual without causing undesirable biological side effects or unwanted interactions. The vehicle is a continuous phase in which the carrier is suspended, and in which excipients may be suspended or dissolved.

In principle, any liquid, semi-solid or soft solid (i.e., a material that could, for example, be swallowed, or used as a lotion base, or as an ointment base) can be used as a NINA vehicle if it is non-aqueous and does not contain an ion concentration sufficient to release the one or more active agents from the carrier. Toxicity requirements are less stringent in topical formulations, so that some common excipients, such as castor oil, are in principle suitable for topical use in much higher concentrations than in formulations intended for oral administration. The carrier-NINA formulations can also be incorporated into bandages, patches, gauze, fabrics, and other macroscopic porous materials for topical application or implantation.

Suitable NINA vehicles include, but are not limited to, plant oils such as sunflower oil, olive oil, peanut oil, corn oil, almond oil, cottonseed oil, sesame oil, soybean oil, canola, oil, castor oil, hydrogenated castor oil, and hydrogenated vegetable oil; animal oils such as fish liver oil and omega 3 lipids; organic solvents that are compatible with tissue, such as glycerol, polyethyleneglycol, and propylene glycol; low molecular weight polyetherpolyols such as polyethylene glycols; mineral oils; silicone oil; semisolid materials, such as cholesterol, ergosterol, lanolin and lanolin alcohols, and petrolatum; lysolipids, phospholipids; crosprovidone; cyclomethinone; dibutyl phthalate; dibutyl sebacate; dimethicone; ethyl oleate; ethylene glycol palmitostearate; glycerin; glyceryl esters such as glycerol behenate, glyceryl monooleate, glyceryl monostearate, and glyceryl palmitostearate; isopropyl alcohol; isopropyl myristate; isopropyl palmitate; lecithin; magnesium stearate and zinc stearate; medium chain triglycerides, poloxomers; polyethylene oxide; polyoxyethylene alkyl ethers; polyoxyethylene castor oil derivatives; polyoxyethylene sorbitan fatty acid esters; polyoxyethylene stearates; propylene carbonate; simethicone; sorbitan esters; higher molecular weight silicones, and other materials used as ointment bases; solid fatty materials, such as tallow and lard; waxes, such as paraffin, beeswax, carnuba wax, microcrystalline wax, and non-ionic and anionic emulsifying wax; hydrophobic resins and gums; fatty alcohols, such as stearoyl alcohol and stearyl alcohol; medium chain alkanes, such as octane, nonane, and decane; derivatives of alkanes, such as aldehydes, sulfonates, esters, ethers, ethoxylates; and combinations thereof.

Semi-solid and solid NINA vehicles are friable and/or flexible, conforming with at most minor application pressure to a topical site, and then releasing active agent, typically by the absorption of moisture from the patient or the atmosphere over the course of hours to days. The NINA vehicle may be a single material, or a mixture of several materials, and may be a solvent for water, immiscible with water, or a combination of these. The NINA vehicle will be essentially non-aqueous, containing water in amounts ranging from about 1% or less, to anhydrous.

If atmospheric moisture, or an aqueous liquid or body fluid, is used to liberate the active agent from a carrier, it may be necessary to add a salt such as NaCl or KCl, in encapsulated form, so that moisture can release the active agent from the carrier.

i. Liquid Suspension

The coated or uncoated active agent-carrier particles can be dissolved or suspended in a NINA vehicle with the composition having (i) an absence of, or very low levels of, ionic ingredients, (ii) a low toxicity, and, optionally for oral administration, (iii) reasonable palatability. Liquid oral dosage forms include nonaqueous solutions, emulsions, suspensions, and solutions and/or suspensions reconstituted from non-effervescent granules, containing suitable solvents, emulsifying agents, suspending agents, diluents, sweeteners, coloring agents, and flavoring agents. Preservatives may or may not be added to the liquid oral dosage forms. Omission of preservatives is favored when possible due to possible allergic reactions to commonly used preservatives.

In preparing the liquid oral dosage forms, the active agent-carrier complexes are incorporated into an orally or topically acceptable NINA vehicle consistent with conventional pharmaceutical practices. The vehicle may include a suitable suspending agent. Known suspending agents include Avicel RC-591 (a microcrystalline cellulose/sodium carboxymethyl cellulose mixture available from FMC), guar gum, alginate, carrageenan, pectin, xanthan, and the like. Such suspending agents are well known to those skilled in the art, and are suitable for use if they are compatible with a particular NINA vehicle. Suitability is readily tested by determining if the suspending agent prevents settling while not significantly affecting the controlled release properties of the coated active agent-loaded carriers.

A liquid suspension can be made by placing the coated, active agent-loaded particles into a liquid NINA vehicle. Surfactants may need to be added to allow dispersion of the coated particles in the oil. Once the oil has mixed with the gastric juices, the loaded carrier particles will be released from the oil and will contact the ionic gastric fluid, and controlled release of the active agent from the particles may commence, or may be delayed until the particles enter the intestine, depending on the coating (if any) applied to the carriers.

For both oral and topical administration, the rate of release of the active agent can be controlled by controlling the water compatibility of the NINA vehicle. For example, a vehicle containing polyethylene glycol or propylene glycol will quickly begin carrying water from the skin and the atmosphere to the active agent-loaded carriers, while a vehicle of isooctane will tend to prevent water access to the carriers until the vehicle has evaporated. A triglyceride vehicle such as olive oil or lard could have an even longer delaying effect, since water would penetrate slowly but the vehicle would not evaporate.

ii. Reconstitutable Dosage Units

Coated active agent-carrier complexes can be formulated into a granular material and packaged in a sachet, capsule or other suitable packaging in unit dose. Such granular material can be reconstituted at the time of use into a suitable NINA vehicle as described above. The granular material may contain excipients that facilitate the dispersion of the particles in the solvent or vehicle used. Formulations of this type have been disclosed in, for example, U.S. Pat. No. 6,077,532, and the manufacture of such unit doses and the use thereof are well known.

iii. Two-Phase Systems

The vehicle, especially for lotion-type topical or intra-gastrointestinal coatings, can be a two phase system of two or more NINA vehicles, usually with surfactant stabilization, or a liposomal formulation. If an ionic material is needed for activation, it can be incorporated directly in the two phase system as a powder, or it can itself be encapsulated with a water-activatable or water-labile coating. Traditional “oil in water” or “water in oil” emulsions, however, are not suitable because of their significant and essential water content.

iv. Soft Gelatin Capsules

A soft gelatin capsule is a one piece hermetically sealed soft gelatin shell containing a liquid, a suspension, a semisolid, or an extruded soft solid. Soft gelatin capsules can be filled with coated or uncoated active agent-loaded carrier particles, or mixtures thereof, suspended in a suitable solution composed of one or more NINA vehicles, or an emulsion of NINA vehicles. The incorporation of a coated active agent-loaded carrier in a NINA vehicle into a soft gelatin capsule provides an easy to swallow dosage form.

In one embodiment, the composition may comprise a first active agent dissolved in a NINA vehicle, and a second active agent bound to a carrier, wherein the active agent/carrier complex is suspended in the oil and the suspension is sealed in a soft gelatin capsule. For example, Lane et al. found that simultaneous administration of anti-emetic compounds of two different classes improved the antiemetic effect of the dosage (J. Pain Symptom Manage. 1991; 6(6): 352-359). In the particular example, a cannabinoid compound Marinol (dronabinol) was administered in an oil in a soft gelatin capsule, while an anti-emetic, prochlorperazine, acting by a different method, was given as a separate pill. Synergistic effects were found.

These two materials (one as a resinate, one dissolved or dispersed in a NINA vehicle) could be combined into a single dosage form. Because prochlorperazine has charged groups, it could be loaded onto a carrier and mixed with the Marinol/oil mixture to unify the two antiemetics in a single-dose formulation. Many synergistic combinations of active agents are mentioned in “Martindale: The Complete Active agent Reference” (Pharmaceutical Press, London), e.g. the 32d edition, and elsewhere in the pharmaceutical literature. The methods of the invention open up new avenues for combining active agents of differing properties in a single formulation.

v. Ointments and Lotions

For topical use, including for localized application in body orifices, the coated active agent/resin particles can be incorporated in a NINA vehicle, preferably a solid or semi-solid, which can then be dispensed to the site. The particles can be formulated to deliver any combination of immediate release and/or coated material that will release on contact with moisture, for example, moisture absorbed from bodily fluids such as sweat, passively effluxed water, exudates from wounds or from mucosa, and other sources of water and/or ionic materials. The container can be of any type, including without limitation ajar, a tube, a hand pump, and an aerosol dispenser. Body moisture will gradually penetrate the preparation and release the active agent. Surfactants can be used to maintain dispersion, and to control the rate of water diffusion into the vehicle. Moreover, as previously described, the vehicle itself can be selected to control the rate of release of the active agent to the tissues of the body.

The benefits of the NINA vehicle and the optionally coated carrier delivery system can apply in these cases as well. The active agent will be protected during storage, and the release can be controlled as opposed to immediate, occurring in parallel with the influx of water and ions. Examples of uses include treatment of the skin and other accessible, moist body cavities, including, for example, the treatment of poison ivy, impetigo, psoriasis, abrasions, bed sores and other ulcerations, candidiasis and other fangal infections, and localized tissue inflammations.

A NINA-carrier material can also be used to provide prophylaxis or treatment for intermittent fluxes of bodily fluids. One example is NINA delivery of an antibiotic for protection and treatment of the skin against the action of digestive juices or urine caused by intermittent stoma leakage from colostomies or uronostomies.

NINA-resinates can also be used to reduce the number of required daily topical applications of an active agent. For example, corticosteroids are typically administered four times a day (for local and systemic effect). The use of NINA resonates may decrease the number of required applications to one to two time a day. The active agent can be supplied as a resinate, or dissolved in the NINA vehicle, or both.

In one embodiment the formulation is used as a combination treatment for psoriasis. Salicylic acid is known as a treatment for removing the flakes of dead skin that are characteristic of psoriasis, and is conventionally sold in lotions, gels, soaps and shampoos for that purpose. Calciopotriene (calciprotriol), a vitamin D derivative, is known to decrease keratinocyte proliferation, to induce keratinocyte differentiation, and to modulate immune responses. It can be used to decrease the production of excess keratinocytes that is characteristic of the underlying condition. Combination of-these two active agents is not currently possible, because the low pH caused by the salicylic acid inactivates the calciopotriene. Using the formulations described herein, these two active agents can be co-delivered by absorbing salicylic acid onto an ion exchange resin or other carrier and optionally coating the resin particles with an aqueous-soluble coating. Then the salicylate-loaded particles can be mixed with an ointment base containing the lipid-soluble calciopotriene, optionally itself encapsulated. Upon application to skin, the calciopotriene will absorb into the tissue and begin to inhibit keratinocyte proliferation, while the salicylate will be released gradually to begin exfoliation of already produced excess skin.

While the above applications have been described in an ointment-type delivery vehicle, any vehicle is potentially suitable, including aerosol or pumped spray, dusting powder, and emulsion. In each of these applications, differences between hydrophilic and hydrophobic vehicles can be used to control delivery rate. Aerosol propellants are typically hydrophobic gases, such as alkanes and haloalkanes, often supplemented with alcohols, and are generally compatible with the NINA vehicles.

B. Excipients

The formulation can contain one or more pharmaceutically acceptable excipients. The one or more excipients can be dissolved or dispersed in the non-ionic, non-aqueous vehicle. Suitable excipients include, but are not limited to, diluents, dispersing agents, solubilzing agents, surfactants, stabilizing agents, pH adjusting agents, flavoring agents, colorants, preservatives, and humectants

III. Combinations of Active Compounds

An active agent loaded onto a carrier, and optionally coated, can be accompanied by other therapeutic entities. Acidic or basic active agents may be administered either as complexes with carriers or as unbound compounds within the final formulation (i.e. dissolved or dispersed within the NINA). These formulations may include, depending on the preparation, additional quantities of the same active agent not absorbed to the carrier, for example for achieving immediate release.

The other entities can also be other active agents, which can be complexed to the carrier or which may be present as particulates or in solution or dispersion, with or without coatings for controlled release. The coating on the active agent-containing carriers may be an extended release coating, taste masking coating, enteric coating, delayed release coating or a combination of these coatings. If the active agent is in the formulation in an unbound form, active agent particles can optionally be coated directly with the various coatings described above.

It is also possible to control the release rates of the various active agents by the selection of different NINA solvents. The rate of water diffusion into the NINA is specific to each NINA, thereby allowing for the controlled release of multiple active agents. This can greatly simplify the development and manufacturing process for controlled release active agents.

IV. Methods of Administration

The formulation can be administered to any patient in need thereof. Although preferred patients are human, animals, especially domestic animals such as dogs, cats, horses, cattle, sheep, goats and fowl, may also be treated with the formulation.

The amount of the active ingredients to be administered is chosen based on the amount which provides the desired dose to the patient in need of such treatment to alleviate symptoms or treat a condition.

Easy-to-swallow formulations are designed to be administered to a patient in need thereof, so that active agent is delivered over approximately 24 hours, although this period may be shortened as needed. The composition improves compliance of patients who have difficulty swallowing by offering an alternative, easy-to-swallow dosage form.

NINA-carrier materials can also be used to deliver active agents, such as antibiotics, to aqueous compartments such as the periodontal pocket. Resin size can be selected to allow washout after dissipation of a NINA vehicle following completion of active agent delivery to the tissue.

Alternatively, the formulation may be delivered topically. Targets of topical delivery include treatment of wounds, or of localized topical conditions, and treatment of non-topical conditions via absorption of an active ingredient through the skin or from an implanted dosage form, as appropriate. The dosage of the active agent in the formulations described herein can be adjusted to suit the patient, which is an advantage over topical devices such as a patch. The formulations can be covered after application with an impermeable bandage if required.

For example, a NINA/carrier formulation can be used to treat shingles caused by herpes virus. Non-narcotic analgesics, such as topical anesthetics (e.g., lidocaine) and/or NSAIDs such as diclofenac are bound to a carrier and applied in substantive NINA lotions, which are easily spread directly on the affected areas. Delivery rate can be adjusted by coatings on the resinates, and by control of NINA polarity and water compatibility. Moreover, the formulations described herein are not easily removed, which is an advantage over patches.

In addition, the formulations described herein are not limited to covering a fixed area or a specific shape. The formulations are flexible and moldable. For example, a long-lasting topical anesthetic for shingles is desirable; but patches for delivering an anesthetic do not conform readily to complexly contoured tissue surfaces, for example the breast or the elbow, while a NINA/carrier ointment is readily applied, and may subsequently be covered by a conventional bandage if needed. Likewise, a NINA/carrier lotion or ointment can be used for treatment of other large area or dispersed conditions, including burns, ulcers, and abrasions. Moreover, it can be difficult to get patches or other macroscopic delivery formats to adhere to mucous membranes (e.g.,rectal, vaginal or oral), or to similar structures such as the sclerum. Semi-solid NINA vehicles, however, could keep a resinate in contact with the mucous membranes for extended periods allowing delivery of the active agent to the desired location.

Extended release topical antibiotics (e.g., antifungals ) that are used to treat infections or prophylactically, e.g., for diaper rash, wounds, or tinea, can be applied in a NINA-carrier form, optionally aerosolized, in conjunction with a bandage or occlusive dressing. For example terbinafine, which is used both topically and orally, is suitable for both topical and oral application as a resinate in a NINA vehicle. Moreover, the active agent could be present in the vehicle as well as on a resin, giving an immediate dose followed by release from the resinate reservoir.

### EXAMPLES

The present invention will be further understood by reference to the following non-limiting examples.

### Example 1

### Preparation of Chlorphenramine Loaded Ion-exchange Resins (Lot 6)

A. Loading of Chlorpheniramine (Maleate salt) to Amberlite IRP-69 (Na-form):

| Ingredient | Quantity/Batch |
|---|---|
| Chlorpheniramine Maleate | 37 g |
| Amberlite IRP-69, Na+ form | 100 g |
| DI Water USP | qs |

Procedure:

Chlorpheniramine was bound to ion exchange resin particles in a single stage binding procedure at room temperature. Briefly, Amberlite IRP-69 resin (100 g) was added to de-ionized water (80 mL). The resulting slurry was well mixed. Chlorpheniramine Maleate (37 g) was added to the resin slurry and subjected to mixing at room temperature for 2 hours to allow binding to occur. The resinate particles were collected by vacuum filtration. The reaction suspension was then filtered using vacuum filtration and washed three times with 1300 mL of de-ionized water. The resulting active agent-resin complex was dried in a forced draft oven at 45° C. until the further loss of water upon complete drying was less than 10% (as measured with a Mettler Toledo Moisture Analyzer at 110° C.).

Active-resin complexes were analyzed for active agent content in the following manner: An accurately weighed, 30 mg sample (for uncoated complexes or coated complexes) was refluxed in 80 mL of an extraction solvent (10% 0.5M sodium acetate in ethanol) for 3 hours. After 3 hours, the mixture was cooled, transferred into a 100 mL volumetric flask with the aid of the extraction solvent, and the volume was brought up to 100 mL with extraction solvent. The resulting solution was analyzed for active agent content via HPLC.

The chlorpheniramine-resin complexes had the following properties:

| | Loss on | Active agent Load |
|---|---|---|
| Lot # | Drying | % chlorpheniramine base on dry basis |
| 6 | 8.70% | 23.1% |

B. Preparation of a Pumpable Ointment Formulation

| Ingredient | Quantity (gm) | Proportion (wt %) |
|---|---|---|
| Castor Oil | 437.5 | 87.5 |
| Hydrogenated Castor Oil | 25 | 5.0 |
| Safflower Oil | 22.5 | 4.5 |
| Polyoxy 10 Oleyl ether | 10 | 2 |
| IR Chlorpheniramine resin | 5 | 1 |

Procedure:

The castor oil was heated to 85° C. The hydrogenated castor oil was added, with stirring, to the castor oil and the mixture was stirred until the hydrogenated castor oil dissolved. The oils were cooled to 40° C., and the safflower oil and polyoxyl 10 oleyl ether emulsifier were added. The mixture was stirred at moderate speed until the mixture was uniform. While stirring at high speed, the chlorpheniramine resin was slowly added, and high speed mixing continued until the resin was fully dispersed. The resin-in-oil dispersion was cooled to room temperature and packaged.

The ointment containing the resin-bound chlorpheniramine can be used as an antihistamine, for example, for the treatment of a local topical inflammation.

### Example 2

### Release Profiles of Albuterol-Carrier Complexes in Different NINA Vehicles

Complexation of Albuterol to an Ion-Exchange Resin

Albuterol is a light-sensitive drug and should be protected from light during analysis. Amberlite IRP-69 was converted to the H+form by placing 100 g dry resin into 1000 g of 3N HCl and incubating the mixture at room temperature for 3 hrs. The resin was recovered on a glass fiber filter in a large Buchner funnel. The resin was washed in the funnel 3 times with 1500 g deionized water, and dried at 45° C. until the “loss on drying” of an aliquot at 110° C. for 1 hour was less than 10% by weight of the resin.

The H+ resin (25 g) was taken up in 250 g deionized water in a beaker and stirred for 15 minutes. The beaker was shielded from light, and then 27 g of albuterol was added to the resin slurry. The mixture was stirred for 3 hours. The resin was collected on a glass fiber filter in a Buchner funnel and rinsed successively with 150 ml DI water, 250 ml DI water, and twice with 200 ml of ethanol. The drug-loaded resin was dried as above.

Albuterol was detected by HPLC on a Waters “Resolve” 5 micron spherical C18 resin column, with a mobile phase of 70% buffer and 30% methanol. Buffer was 4.4 g 1-heptane sulfonic acid in 100 g DI water, adjusted to pH 3.2±0.1 with glacial acetic acid. Albuterol extraction solvent was 10% v/v 0.5 M Na Acetate in ethanol. A stock solution of 0.5 mg albuterol/ml extraction buffer was diluted with 25 volumes of “diluent” (30:70 methanol:DI water, v:v) at the time of use. Samples were extracted to determine albuterol content by transferring 200 mg albuterol resin to a 250 ml flask, using extraction solvent; bringing the volume to about 220 ml of extraction solvent; shaking overnight at room temperature; adding extraction solvent to 250 ml; filtering an aliquot; and diluting filtered solution with 24 volumes of diluent. This solution was analyzed by HPLC, and the amount was determined by comparison with a chromatogram of the standard.

Release of Albuterol from Resinate in Various Vehicles

A system was developed to simulate the effects of topical application of a resin-bound drug in a vehicle to skin. A multiwell-type tissue culture system was used to maintain resinate inside a membrane-bottomed insert (Falcon brand part no.35-3090) in contact with a reservoir of aqueous solution, which was separated from the inside of the well by a microporous membrane (in this case, 0.4 micron pores in track-etched PET.) The inserts were floated on a water surface in an array (Falcon 35-3502) of independent wells containing a fixed amount of aqueous solution, one insert per well. The wells were shaken at room temperature using a rotary shaker set to 50 rpm to stir the solution in the wells.

The experimental design allowed the pores of the membrane to fill with the aqueous solution. Water would then dissolve in the NINA vehicle, and exchange of drug between the resin and the water in the vehicle would occur. Finally, the drug would migrate through the membrane pores. By this route, drug would gradually build up in the bulk solution in the well. Wells were sampled at various times and the fluid was analyzed for albuterol. Four conditions were tested, using the same batch of albuterol resinate.

a. Release in Phosphate Buffer.

7.5 g phosphate buffer (pH 6.8; 0.05 M) was added to each well. 17.29 mg of albuterol resinate was added to each of 48 inserts. The inserts were placed in the wells of six well trays, and the assembled plates were placed on an orbital shaker at low speed (ca. 50 RPM). Two 6 well trays were removed for analysis at 0.5, 1, 2 and 4 hours. The inserts were removed, and the fluid in the wells (12 in all) was analyzed for albuterol by HPLC.

b. Release in Phosphate Buffer with Dipropylene Glycol/Carbomer Vehicle.

Carbomer (polyacrylic acid; Carbopol 934P from Noveon) was dissolved in dipropylene glycol to form a 1% w/w solution. The carbomer was intended to viscosify the dipropylene glycol; it may also have interacted with the drug. Resinate (1038 mg) was dispersed in 30 g of 1% carbomer/dipropylene glycol solution. Each of 48 inserts received 0.5 g of the dispersion. 7.5 g of phosphate buffer, pH 6.8, was added to each well. The inserts were placed in the wells, and the experiment was conducted as described above.

c. Release in Phosphate Buffer with Castor Oil/Silica/Surfactant Vehicle.

Castor oil (30 g) was viscosified with colloidal silica (2.5%) [Cab-o-Sil M5P; Cabot] and 5% oleth-10 surfactant (Volpo 10; Croda) was added to form the vehicle. 1038 mg of albuterol resinate was added and dispersed in the vehicle. 0.5 g of this dispersion was dispensed into each of 48 inserts, and phosphate buffer pH 6.8 into each of 48 wells. The inserts were placed in the wells, and the experiment was conducted as described above.

d. Release in Phosphate Buffer with Castor Oil/Silica Vehicle.

Castor oil (30 g) was viscosified with colloidal silica (2.5%) to form the vehicle; no surfactant was added. 1038 mg of albuterol resinate was added and dispersed in the vehicle. 0.5 g of this dispersion was dispensed into each of 48 inserts, and phosphate buffer pH 6.8 into each of 48 wells. The inserts were placed in the wells, and the experiment was conducted as described above.

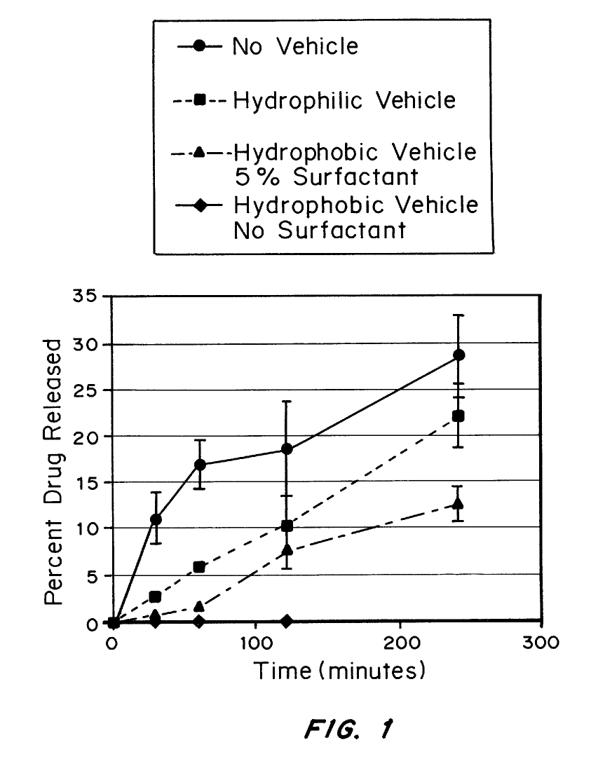

The results of these experiments are shown in FIG. 1. Release of albuterol was fastest in the ionic aqueous solution; and slowest (not detectable) in the hydrophobic castor oil vehicle without surfactant. Thus, it is demonstrated experimentally that NINA vehicles can be selected to transport a hydrophilic drug from a resinate to a tissue mimetic at a selected rate in the presence of trace moisture, as might be obtained from skin, or from the air.

These experiments demonstrate that the rate of release of a drug, such as albuterol, from an ion exchange resin can be controlled by selection of the NINA vehicle, and by selection of the appropriate surfactants and/or other excipients present in the vehicle. The experiments also demonstrate that an active agent, such as a hydrophilic drug, is not eluted from the carrier until moisture penetrates into the vehicle, or water is solubilized in the vehicle by surfactants. The release of the drug from the carrier is a controlled release, resulting in a gradual release rather than a sudden burst. These release patterns are obtained, in this example, without coating the agent-loaded carrier with any kind of coating. This can be a significant simplification in the manufacturing process.

Moreover, although carriers have in some cases been included in ointments, this is believed to be the first demonstration that simple systems of this sort can provide a controlled rate of release of an active agent, as opposed to immediate release, into the skin or other organs.

We claim:

1. A composition for delivery of an active agent, the composition comprising one or more active agents complexed to one or more carriers,
wherein the carrier/active agent complexes are optionally coated with a polymeric coating selected from the group consisting of extended release coatings, delayed release coatings, immediate release coatings, and combinations thereof, to form particles, and
wherein the particles are dispersed in a non-ionic, non-aqueous vehicle.

2. The composition of claim 1 wherein the active agent is selected from the group consisting of analeptic agents; analgesic agents; anesthetic agents; antiasthmatic agents; antiarthritic agents; anticancer agents; anticholinergic agents; anticonvulsant agents; antidepressant agents; antidiabetic agents; antidiarrheal agents; antiemetic agents; antihelminthic agents; antihistamines; antihyperlipidemic agents; antihypertensive agents; anti-infective agents; anti-inflammatory agents; antimigraine agents; antineoplastic agents; antiparkinsonism active agents; antipruritic agents; antipsychotic agents; antipyretic agents; antispasmodic agents; antitubercular agents; antiulcer agents; antiviral agents; anxiolytic agents; appetite suppressants (anorexic agents); attention deficit disorder and attention deficit hyperactivity disorder active agents; cardiovascular agents including calcium channel blockers and antianginal agents; central nervous system (“CNS”) agents; beta-blockers and antiarrhythmic agents; central nervous system stimulants; diuretics; genetic materials; hormonolytics; hypnotics; hypoglycemic agents; immunosuppressive agents; muscle relaxants; narcotic antagonists; nicotine; nutritional agents; parasympatholytics; peptide active agents; psychostimulants; sedatives; sialagogues, steroids; smoking cessation agents; sympathomimetics;. tranquilizers; vasodilators; beta-agonist; tocolytic agents; and combinations thereof.

3. The composition of claim 1 wherein the carrier is an ion-exchange resin.

4. The composition of claim 1 wherein the non-ionic, non-aqueous vehicle is selected from the group consisting of almond oil, canola oil, castor oil, hydrogenated castor oil, cetosrearyl alcohol, cetyl alcohol, cholesterol, corn oil, cotton seed oil, crospovidone, cyclomethicone, dibutyl phthalate, dibutyl sebacate, dipropylene glycol, dimethicone, ergosterol, ethyl oleate, ethylene glycol palmitostearate, fish oil, glycerin, glyceryl behenate, glyceryl monooleate, glyceryl monostearate, glyceryl palmitostearate, butane, propane, isobutene, isopropyl alcohol, isopropyl myristate, isopropyl palmitate, lanolin, lanolin alcohols, lecithin, magnesium stearate, medium chain triglycerides, mineral oil, light mineral oil, olive oil, omega-3 oils, paraffin, peanut oil, petrolatum, poloxamers, polyethylene glycol, polyethylene oxide, polyoxyethylene alkyl ethers, polyoxyethylene castor oil derivatives, polyoxyethylene sorbitan fatty acid esters, polyoxyethylene stearates, propylene carbonate, propylene glycol, sesame oil, shark oil, simethicone, silicon oils, sorbitan esters, soybean oil, stearyl alcohol, sunflower oil, suppository base hard fat, triacetin, tributyl citrate, triethyl citrate, vegetable oil, vegetable oil hydrogenated, vitamin E, wax anionic emulsifying, wax carnuba, wax cetyl esters, wax microcrystalline, wax nonionic emulsifying, wax white (beeswax), wax yellow (natural beeswax), zinc stearate, ergosterol, lysolipids, phospholipids, tallow, lard, waxes, hydrophobic solid resins and gums, fatty alcohols, and combinations thereof.

5. The composition of claim 1 wherein the non-ionic, non-aqueous vehicle is a liquid, solid or semisolid.

6. The composition of claim 1 wherein the composition is formulated for topical or oral administration.

7. The composition of claim 1 wherein the composition is formulated for topical administration.

8. The composition of claim 7 wherein the composition is in a form selected from the group consisting of a spray, an aerosol, a lotion, a pumpable lotion, an ointment, a liposomal composition, a suppository, a gel, and a material impregnated into a bandage, mesh, fabric or a patch.

9. The composition of claim 8 wherein the composition in the form of a spray or aerosol which can be administered in a metered or unmetered dose.

10. The composition of claim 1 wherein the composition is formulated for oral administration.

11. The composition of claim 10 wherein the composition is in a form selected from the group consisting of hard gelatin capsules, tablets, chewable tablets, powders, solutions, suspensions, sachets, soft gelatin capsules, molded chewable objects.

12. The composition of claim 11 wherein the composition is encapsulated in a soft gelatin capsule.

13. The composition of claim 1 further comprising one or more excipients.

14. The composition of claim 1 wherein at least a first active agent is complexed to the carrier and a second active agent is dissolved or dispersed in the non-ionic, non-aqueous vehicle.

15. The composition of claim 1 wherein the carrier is less than about 150 microns in diameter.

16. The composition of claim 1 wherein the coating is formed from an aqueous dispersion of a synthetic polymer.

17. The composition of claim 16 wherein the coating is formed from an aqueous dispersion of a methacrylic ester co-polymer.

18. The composition of claim 17 wherein the coating level is greater than 5% by weight.

19. The composition of claim 1 wherein the coating is an extended release coating and the active agent is present in an amount of less than about 35% by weight if the carrier is irregular in shape and less than about 28% if the carrier is regular in shape.

20. The composition of claim 1 wherein the complexes are taste-masked particles, prepared by coating active agent-carrier complex with a polymer that is insoluble in the neutral environment of saliva, but dissolves in the acid environment of the stomach.

21. The composition of claim 1 wherein the active agent-carrier are coated with a polymer that is mucoadhesive in the oral cavity.

22. The composition of claim 1 providing an extended release of active agent to produce a therapeutic effect over approximately 24 hours.

23. The composition of claim 1 providing an extended release of active agent to produce a therapeutic effect over approximately 12 hours.

24. The composition of claim 1 wherein the particles comprise
less than about 50% by weight active agent and an extended release polymeric coating on the particles,
wherein the coating material is applied to the active agent-complexes from an aqueous dispersion.

25. The composition of claim 1 comprising particles comprising an immediate release coating and a delayed release coating.

26. The composition of claim 25 wherein the immediate release coating is a taste masking coating.

27. The composition of claim 25 wherein the immediate release coating is a mucoadhesive coating.

28. The composition of claim 1 comprising particles which have different coatings or wherein some active agent-carrier complexes are uncoated and some are coated.

29. The composition of claim 28 providing pulsatile release.

30. The composition of claim 1 wherein the delayed release coating is an enteric coating.

31. The composition of claim 1 wherein the composition is substantially anhydrous.

32. The composition of claim 31 wherein the formulation contains less than 1% water by weight.

34. A method of delivering an active agent comprising administering to idual in need thereof the composition of claim 1.

35. The method of claim 34 wherein the composition is administered .

36. The method of claim 34 wherein the composition is administered 37. The method of claim 34 wherein the formulation is in a bandage, auze, mesh or fabric and is applied topically or by implantation.
